(19) United States
(12) Patent Application Publication
(10) Pub. No.: US 2002/0055465 A1
(43) Pub. Date: May 9, 2002

(54) Nocathiacin antibiotics prepared by biotransformation or chemical methods

(76) Inventors: Wenying Li, Middletown, CT (US); John Leet, Madison, CT (US); Xiaohua Huang, Madison, CT (US); Kin Sing Lam, North Haven, CT (US); Alicia Regueiro-Ren, Middletown, CT (US)

Correspondence Address: MARLA J MATHIAS, BRISTOL-MYERS SQUIBB COMPANY, PATENT DEPARTMENT, P O BOX 4000, PRINCETON, NJ, 08543-4000, US

(21) Appl. No.: 09/919,455

(22) Filed: Jul. 31, 2001

Related U.S. Application Data
(60) Provisional application No. 60/225,114, filed on Aug. 14, 2000.

(51) Int. Cl. A61K 38/14; C07H 17/02; C07K 9/00; A61K 31/70

(52) U.S. Cl. 514/8; 530/322; 536/17.4; 514/23

(57) ABSTRACT

Novel nocathiacin derivatives have been prepared by both biotransformation and chemical methods. A novel nocathiacin derivative, nocathiacin IV, was prepared by either incubation of protease from Streptomyces griseus (Sigma, Cat# P5147) with nocathiacin I in DMF or by chemical methods. Fermentation of Actinoplanes sp. ATCC 53771 in the presence of nocathiacin IV produces a new compound nocathiacin IV 6-deoxyglucoside. Both nocathiacin IV and nocathiacin IV 6-deoxyglucoside have broad spectrum antibiotic activity against Gram-positive bacteria and have in vivo efficacy in animals.

### CROSS REFERENCE TO RELATED APPLICATIONS

This application claims the benefit of U.S. Provisional Application Serial No. 60/225,114 filed Aug. 14, 2000.

### FIELD OF THE INVENTION

This invention relates to novel nocathiacin derivatives useful for the treatment of serious bacterial infections and suitable for both oral and parenteral administration. This invention also relates to a pharmaceutical composition, especially an antibacterial composition, which comprises the novel nocathiacin derivative as an active ingredient. The invention also provides a method for treating serious bacterial infections by administering to a mammal in need thereof said nocathiacin derivatives or a pharmaceutical composition of the nocathiacin derivatives. Additionally, this invention provides processes, by both biotransformation and chemical methods, for the preparation of the novel nocathiacin derivatives.

### BACKGROUND OF THE INVENTION

Multidrug-resistant strains of many clinically important pathogenic bacteria, including methicillin-resistant Staphylococcus aureus (MRSA), Streptococcus pneumoniae, Mycobacterium tuberculosis, and Enterococci strains are becoming a worldwide health problem. There is an urgent need to discover new agents to treat patients infected with multidrug-resistant bacteria. A new group of thiazolyl peptide antibiotics, (designated herein as nocathiacins) having inhibitory activity at the nanomolar level against Gram-positive bacteria has been discovered. The present invention relates to novel antibiotic compounds, such as nocathiacin IV, nocathiacin IV 6-deoxyglycoside and comprising processes for preparing them from a nocathiacin by either biotransformations with biocatalysts or by chemical methods. The novel nocathiacin products described herein exhibit potent antimicrobial activity against Gram-positive bacteria in vitro. These compounds and their precursors, nocathiacin I and nocathiacin III, are antibiotics useful in the treatment of bacterial infections in humans.

Nocathiacins I and III have been previously described by J. E. Leet et al in PCT WO 00/03722, published Jan. 27, 2000, commonly owned by Applicant herein; and Nocathiacin I in Sasaki, T. et al, J. of Antibiotics 51, No. 8, pp. 715-721 (published Aug. 25, 1998). Other nocathiacin analogs are described in W. Lee et al PCT WO 00/14100 published Mar. 16, 2000. The novel nocathiacin antibiotics of this invention are related to but clearly distinguishable from nosiheptide (Prange T. et al, J. Am Chem Soc. 99, 6418 (1977); Benazet, F. et al Experientia 36, 414 (1980); Floss, H. G. et al, J. Am Chem Soc. 115, 7557 (1993); glycothiohexides (Steinberg, D. A. et al, J. Antibiot. 47, 887 (1994); M. D. Lee et al, J. Antibiot. 47, 894 (1994); M. D. Lee et al, J. Antibiot. 47, 901 (1994); U.S. Pat. No. 5,451,581, 1995), and Antibiotic S-54832A (U.S. Pat. No. 4,478,831, 1984).

### SUMMARY OF THE INVENTION

A first embodiment of a first aspect of the present invention are compounds of Formula I

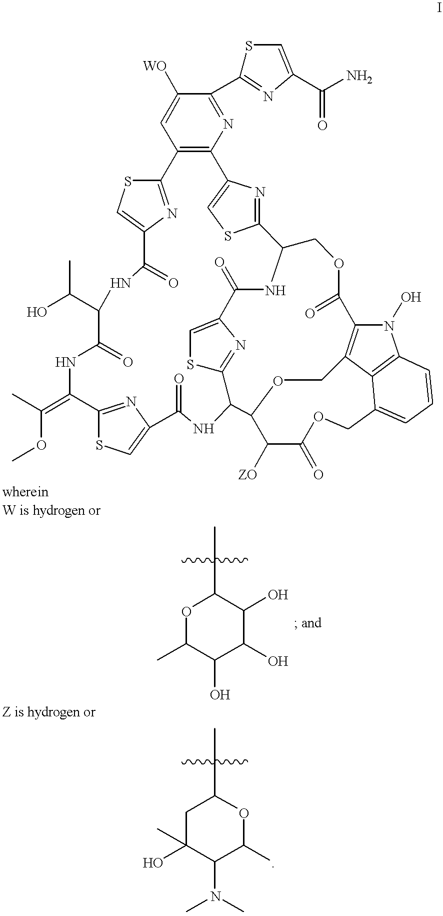

wherein

W is hydrogen or

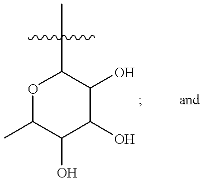

Z is hydrogen or

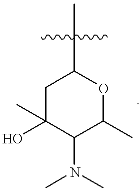

A first embodiment of a second aspect of the present invention is an isolated and substantially pure form of nocathiacin IV, or a pharmaceutically acceptable salt thereof, wherein the antibiotic has the following characteristics:

(a) its hydrochloride salt appears as a shiny colored granular solid;

(b) has a molecular weight of 1367 as determined by mass spectrometry;

(c) has the molecular formula C₅₈H₅₇N₁₃O₁₇S₅

(d) its hydrochloride salt exhibits an ultraviolet absorption spectrum when dissolved in methanol substantially as shown in FIG. 1;

(e) its hydrochloride salt exhibits an infrared absorption spectrum (KBr) substantially as shown in FIG. 2;

(f) when dissolved in deuterated dimethylsulfoxide, its hydrochloride salt exhibits a proton magnetic resonance spectrum substantially as shown in FIG. 3;

(g) when dissolved in deuterated dimethylsulfoxide, its hydrochloride salt exhibits a ¹³C magnetic resonance spectrum substantially as shown in FIG. 4;

(h) exhibits a high performance liquid chromatography retention time of 8.8 minutes with a C18 reversed phase silica gel column using a 1 mM hydrochloric acid—acetonitrile gradient as described in the analytical HPLC section;

(i) and has the formula

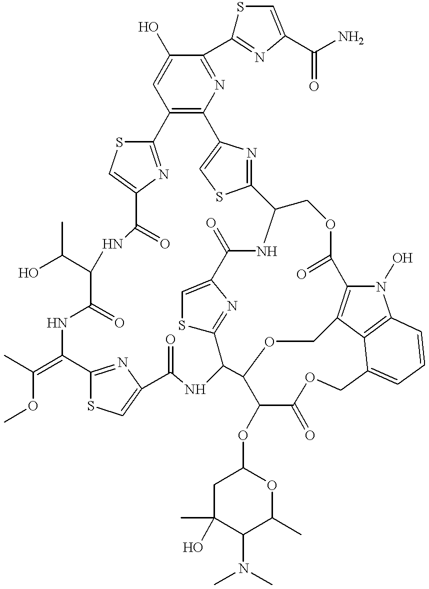

A second embodiment of the second aspect of the present invention is the compound of the first embodiment of the second aspect wherein the pharmaceutically acceptable salt is the hydrochloride salt.

A first embodiment of a third aspect of the present invention is an isolated and substantially pure form of nocathiacin IV 6-deoxyglucoside, or a pharmaceutically acceptable salt thereof, wherein the antibiotic has the following characteristics:

(a) its hydrochloride salt appears as a spongy colored amorphous solid;

(b) has a molecular weight of 1513 as determined by mass spectrometry;

(c) has the molecular formula C₆₄H₆₇N₁₃O₂₁S₅

(d) its hydrochloride salt exhibits an ultraviolet absorption spectrum when dissolved in methanol substantially as shown in FIG. 5;

(e) its hydrochloride salt exhibits an infrared absorption spectrum (KBr) substantially as shown in FIG. 6;

(f) when dissolved in deuterated dimethylsulfoxide, its hydrochloride salt exhibits a proton magnetic resonance spectrum substantially as shown in FIG. 7;

(g) when dissolved in deuterated dimethylsulfoxide, its hydrochloride salt exhibits a 13C magnetic resonance spectrum substantially as shown in FIG. 8;

(h) exhibits a high performance liquid chromatography retention time of 7.5 min with a C18 reversed phase silica gel column using a 1 mM hydrochloric acid—acetonitrile gradient as described in the analytical HPLC section;

(i) and has the formula

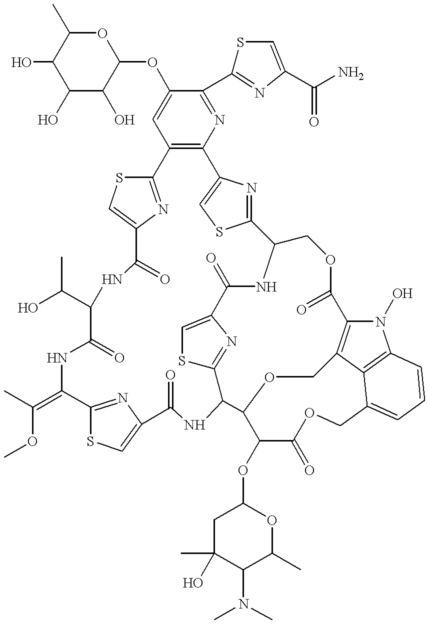

A second embodiment of a third aspect of the present invention is the compound of the first embodiment of the third aspect wherein the pharmaceutically acceptable salt is the hydrochloride salt.

A first embodiment of a fourth aspect of the present invention is a pharmaceutical composition comprising a therapeutically effective amount of a compound as described in any of the first through third aspects of the invention and a suitable carrier, adjuvant or diluent.

A first embodiment of a fifth aspect of the present invention is a method for preventing or treating infection of a mammal by a bacterium, comprising the step of administering a therapeutically effective amount of a compound or composition as described in the first through fourth aspects to said mammal in need thereof.

A first embodiment of the sixth aspect of the present invention is a method for making nocathiacin IV from nocathiacin I comprising the steps of converting nocathiacin I to nocathiacin IV in a DMF solution containing protease from Streptomyces griseus (Sigma, Cat# P5147), and isolating said nocathiacin IV from the biotransformation reaction mixture.

A second embodiment of the sixth aspect of the present invention, which depends from the first embodiment of the sixth aspect, is a method for making nocathiacin IV from nocathiacin I comprising the steps of converting nocathiacin I to nocathiacin IV in an appropriate solvent containing a mineral acid and an alkyl halide, and isolating said nocathiacin IV from the chemical reaction mixture.

A third embodiment of the sixth aspect of the present invention, which depends from the first embodiment of the sixth aspect, is a method for making nocathiacin IV 6-deoxyglucoside from nocathiacin IV comprising the steps of converting nocathiacin IV to nocathiacin 6-deoxyglucoside in a biotransformation broth containing fermented culture of microorganism Actinoplanes sp. (ATCC-53771); and isolating said nocathiacin IV 6-deoxyglucoside from the biotransformation reaction broth.

The description of the invention herein should be construed in congruity with the laws and principals of chemical bonding. An embodiment or aspect which depends from another embodiment or aspect, will describe only the variables having values and provisos that differ from the embodiment or aspect from which it depends. Thus, for example, an embodiment which reads “the compound of formula I according to the n^{th} aspect of the invention, wherein Z is hydrogen,” should be read to include all remaining variables with values defined in the n^{th} aspect and should be read to further include all the provisos, unless otherwise indicated, pertaining to each and every variable in the n^{th} aspect.

### BRIEF DESCRIPTION OF THE FIGURES

FIG. 1: UV spectrum of nocathiacin IV hydrochloride salt

FIG. 2: IR spectrum of nocathiacin IV hydrochloride salt

FIG. 3: ¹H-NMR spectrum (500 MHz) of nocathiacin IV hydrochloride salt in deuterated dimethylsulfoxide

FIG. 4: ¹³C-NMR (125 MHz) spectrum of nocathiacin IV hydrochloride salt in deuterated dimethylsulfoxide

FIG. 5: UV spectrum of nocathiacin IV 6-deoxyglucoside hydrochloride salt

FIG. 6: IR spectrum of nocathiacin 6-deoxyglucoside hydrochloride salt

FIG. 7: ¹H-NMR spectrum (500 MHz) of nocathiacin IV 6-deoxyglucoside hydrochloride salt in deuterated dimethylsulfoxide

FIG. 8: ¹³C-NMR (125 MHz) spectrum of nocathiacin IV 6-deoxyglucoside hydrochloride salt in deuterated dimethylsulfoxide

### DESCRIPTION OF THE INVENTION

The term “alkyl halide” as used herein and in the claims (unless specified otherwise) means a straight or branched chain halogenated alkane such as methyl iodide, ethyl bromide, propyl iodide, isopropyl iodide and the like.

The term “mineral acid” as used herein means hydrochloric acid, hydrobromic acid and hydroiodic acid.

The following abbreviations, most of which are conventional abbreviations well known to those skilled in the art, are used throughout the description of the invention and examples. Some of the abbreviations used are as follows:

| | |
|---|---|
| h | = hour(s) |
| rt = | = room temperature |
| mol = | = mole(s) |
| mmol = | = millimole(s) |
| kg = | = kilogram(s) |
| g = | = gram(s) |
| mg = | = milligram(s) |
| L = | = liter(s) |
| mL = | = milliliter(s) |
| BHA = | = 3-tert-Butyl-4-hydroxyanisole |
| BHT = | = 2,6-Di-tert-butyl-4-methylphenol |
| CFU = | = colony forming unit |
| DMF = | = N,N-Dimethylformamide |
| DMSO = | = Dimethylsulfoxide |
| HC1 = | = Hydrochloric Acid |
| THF = | = Tetrahydrofuran |
| TFA = | = Trifluoroacetic acid |

The present invention describes novel derivatives of nocathiacin antibiotics obtained through microbial biotransformation or by chemical methods. The invention provides an efficient method for the preparation of nocathiacin IV and nocathiacin IV 6-deoxyglucoside from nocathiacin I. One of the novel processes of this invention comprises biotransformation of nocathiacin I with protease from Streptomyces griseus (Sigma, Cat# P5147), and fermentation of Actinoplanes sp. ATCC 53771, Amycolata autotrophica ATCC 35204, or mutants thereof in the presence of substrate nocathiacin IV in a nutrient medium, and isolation of the resulting biotransformation products, nocathiacin IV and nocathiacin IV-6-deoxyglucoside, respectively, in a conventional manner. Another novel process of this invention comprises treating nocathiacin I with a mineral acid and an alkyl halide in an appropriate solvent and isolating nocathiacin IV from this reaction mixture. It will be appreciated by one skilled in the art that these transformations will also be applicable to the other nocathiacin starting materials described herein, such as nocathiacin III.

### Biotransformation

The nocathiacins used as starting materials herein have the general structural formula:

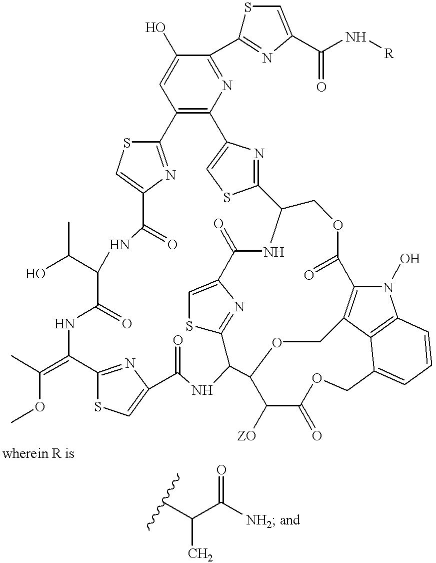

wherein R is

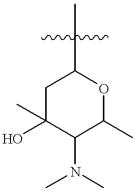

Z is hydrogen (for nocathiacin III) or

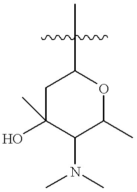

(for nocathiacin I).

The preparation of starting materials nocathiacin antibiotics I and III may be carried out by cultivating Nocardia sp. (ATCC-202099) in a suitable nutrient medium under conditions described herein, preferably under submerged aerobic conditions, until a substantial amount of nocathiacins are detected in the fermentation, harvesting by extracting the active components from the mycelial growth with a suitable solvent, concentrating the solution containing the desired components, then subjecting the concentrated material to chromatographic separation to isolate the compounds from other metabolites also present in the cultivation medium.

Production of nocathiacin can be effected at temperature conducive to satisfactory growth of the producing organism, e.g. 16° C. and 40° C., but it is preferable to conduct the fermentation at 22° C. to 32° C. The aqueous medium is incubated for a period of time necessary to complete the production of nocathiacin as monitored by high pressure liquid chromatography (HPLC) usually for a period of about 1-6 days, on a rotary shaker operating at about 50 rpm to 300 rpm, preferably at 150 rpm to 250 rpm.

The preparation of nocathiacin I and III starting materials is described more fully in commonly-owned U.S. application Ser. No. 09/351,791 filed Jul. 13, 1999 to Leet, et al, which is hereby incorporated by reference.

The R group of nocathiacin I was removed by either treatment with an enzyme or microorganism or by chemical methods to give nocathiacin IV. Nocathiacin IV was then glycosylated by treatment with an enzyme or microorganism to yield nocathiacin IV 6-deoxyglucoside.

The novel nocathiacin derivatives thus obtained by the described processes have the structural formula:

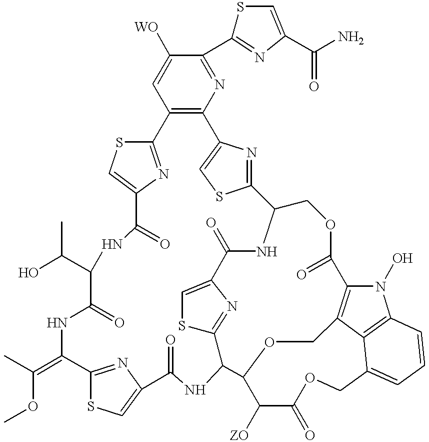

wherein W is hydrogen or

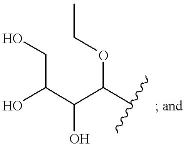

and

Z is hydrogen or

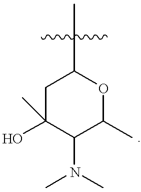

The enzyme, protease from Streptomyces griseus (Sigma, Cat# P5147), employed in the present invention may also be any enzyme or microorganism capable of converting nocathiacin I to nocathiacin IV. It is appreciated by one skilled in the art that the same enzymatic transformation may also be carried out using nocathiacin III as substrate. The enzyme or microbial materials, regardless of origin or purity, may be employed in the free state or immobilized on a support such as by physical adsorption or entrapment. The reaction may be carried out in aqueous solvent or organic solvent or a solvent mixture, while a preferred solvent is N,N-dimethylformamide. The substrate concentration may be between 0.5 to 10 g/L. The enzyme to substrate ratio may be between 0.2:1 w/w to 10:1 w/w. The reaction mixture was incubated on a rotary shaker operating at about 250 rpm at a temperature between 24° C. and 32° C., preferably at 27° C., and on a rotary shaker operating at between 100 to 250 rpm. The reaction was completed in 1 to 3 days

The microorganisms, Actinoplanes sp. ATCC 53771 employed in the present invention may also be any microorganism capable of converting nocathiacin IV to nocathiacin IV 6-deoxyglucoside. It is appreciated by one skilled in the art that the same glycosylation may also be carried out using nocathiacin III derived compounds as substrate. The microorganism, regardless of origin or purity, may be employed in the free state or immobilized on a support such as by physical adsorption or entrapment. The preferred biotransformation microorganisms that were used in this study for conversion of nocathiacin to nocathiacin 6-deoxyglucoside were obtained from American Type Culture Collection. The taxonomic analysis of Actinoplanes sp. ATCC 53771 has been described in U.S. Pat. No. 4,981,792 (Jan. 1, 1991).

In general, nocathiacin IV 6-deoxyglucoside can be produced by incubating the aforementioned microorganism cell in the presence of an appropriate concentration of substrate nocathiacin IV in an aqueous medium, which may contain pH buffering material, sources of assimilable carbon, or sources of nitrogen, preferably under submerged aerobic conditions.

The aqueous medium is incubated at a temperature between 24° C. and 32° C., preferably at 28° C. The aqueous medium is incubated for a period of time necessary to complete the biotransformation as monitored by high pressure liquid chromatography (HPLC) usually for a period of about 20-48 hours after the addition of the substrate, on a rotary shaker operating at about 180 rpm to 250 with a throw of about 2 inches.

Growth of the microorganisms may be achieved by one of ordinary skill of the art by the use of appropriate medium. Appropriate media for growing microorganism include those which provide nutrients necessary for the growth of microbial cells. A typical medium for growth includes necessary carbon sources, nitrogen sources, and trace elements. Inducers may also be added. The term inducer as used herein, includes any compound enhancing formation of the desired enzymatic activity within the microbial cell.

Carbon sources may include sugars such as glucose, fructose, galactose, maltose, sucrose, mannitol, sorbital, glycerol starch and the like; organic acids such as sodium acetate, sodium citrate, and the like; and alcohols such as ethanol, propanol and the like.

Nitrogen sources may include N—Z amine A, corn steep liquor, soybean meal, beef extract, yeast extract, tryptone, peptone, cottonseed meal, peanut meal, amino acids such as sodium glutamate and the like, sodium nitrate, ammonium sulfate and the like.

Trace elements may include magnesium, manganese, calcium, cobalt, nickel, iron, sodium and potassium salts. Phosphates may also be added in trace or preferably, greater than trace amounts.

The medium employed may include more than one carbon or nitrogen source or other nutrient. Preferred media for growth include aqueous media, particularly that described in the example herein.

### Isolation and Structural Characterization

The products, such as nocathiacin IV and nocathiacin IV 6-deoxyglucoside, can be recovered from the reaction solvent or culture medium by conventional means which are commonly used for the recovery of other known biologically active substances. Accordingly, crude nocathiacin IV can be obtained upon removal of enzyme by centrifugation or filtration and removal of reaction solvent by evaporation; while crude nocathiacin IV 6-deoxyglucoside can be obtained upon extraction of the reaction culture with a conventional solvent, such as ethyl acetate. The purified products can then be obtained upon treatment with a conventional resin (e.g. anion or cation exchange resin, non-ionic adsorption resin), treatment with a conventional adsorbent (e.g. activated charcoal, silica gel, cellulose, alumina), crystallization, recrystallization, and/or purification by reverse phase preparative HPLC. The purification of the novel nocathiacin antibiotic products was monitored using analytical HPLC. The structure of nocathiacin derivatives, shown previous, was assigned based on 2D NMR studies and positive ion electrospray HRMS and MS/MS data.

### Analytical HPLC

The synthesis and purification of the nocathiacin biotransformation products was monitored by HPLC analysis on an YMC Packed ODS-AQ column, 4.6 mm i.d.×15 cm 1. Analyses were done on a Hewlett Packard 1090 Series Liquid Chromatograph, with UV detection at 220 and 360 nm. A gradient system of 1 mM HCl (solvent A) and acetonitrile (solvent B) was used: 15%B, 3 min; 15% to 40%B linear gradient, 1 min; 40%B, 5 min; 40% to 85% linear gradient, 1 min; 85%, 1 min; 85% to 15%B linear gradient, 1 min. The eluant was pumped at a flow rate of 1.2 ml/min.

### Preparative HPLC

The following components were used to construct a preparative HPLC system: Beckman Instruments Inc. (Somerset, N.J.), Beckman “System Gold” 126 Programmable Solvent Module; Beckman 168 Diode Array Detector Module; Beckman “System Gold” Version 711U software; Preparative HPLC column: YMC Inc. (Wilmington, N.C.) preparative column, fitted with a ODS-A 25μ particle size, 120 Å pore size, 10 mm i.d.×10 mm 1. drop-in guard module; mobile phase: 1 mM HCl—acetonitrile; UV detection:330 nm.

### Chemistry

The compounds of the present invention may also be obtained by chemical methods. As shown in Step 1 of Scheme 1, below, an appropriate nocathiacin, such as nocathiacin I or nocathiacin III, may be treated with a mineral acid, HX (X=Cl, Br, I), and an alkyl halide in an appropriate solvent within the temperature range of 20° C. to 80° C. The mineral acid, HX, includes hydrochloric acid, hydrobromic acid and hydroiodic acid, with the most preferred mineral acid being hydroiodic acid. The most preferred alkyl halide was found to be methyl iodide. Appropriate solvents include, but are not limited to, THF, DMF, dioxane, and DMSO with the most preferred solvent being THF. The reaction may be carried out between 20° C. to 80° C., with the preferred temperature range being 40° C. to 50° C. The resulting product, wherein the dehydroalanine moiety of the corresponding starting material has been removed, may be precipitated from the reaction mixture by addition of an appropriate solvent, such as diethyl ether. The crude product may then be collected by filtration and dried under vacuum. The crude product can be further purified by reverse phase chromatography on a preparative C-18 column using acetonitrile/water/trifluoroacetic acid or methanol/water/trifluoroacetic acid as the mobile phase. The product containing fractions of sufficient purity, as determined by analytical HPLC, may be combined and concentrated under reduced pressure to remove volatiles. The resultant solution may then be frozen and lyophilized to provide the purified product as the trifluoroacetic acid salt. Other dilute aqueous acids, such as dilute hydrochloric acid, could be used in place of the trifluoroacetic acid in the mobile phase, and would provide the purified product as the corresponding salt form.

The nocathiacin products obtained as a salt form may be converted to the corresponding free base by methods known to one skilled in the art. For example, the nocathiacin product (as a salt form) may be dissolved in an appropriate solvent and treated with an appropriate resin bound base. Appropriate solvents include, but are not limited to, CH₃CN, MeOH and THF. An appropriate resin bound base is 1, 3-2-diazaphosphorine on polystyrene, although other resin bound bases may be employed. The mixture may then be filtered, the filter cake washed with appropriate solvents and water, and the filtrate concentrated under reduced pressure to remove volatiles. The resulting solution may then be frozen and lyophilized to provide the nocathiacin product, such as nocathiacin IV, as the free base. The product of Step 1 of Scheme 1 may be further elaborated by glycosylation according to the conditions previously described in the biotransformation section. The resultant glucosides may then be purified as previously described.

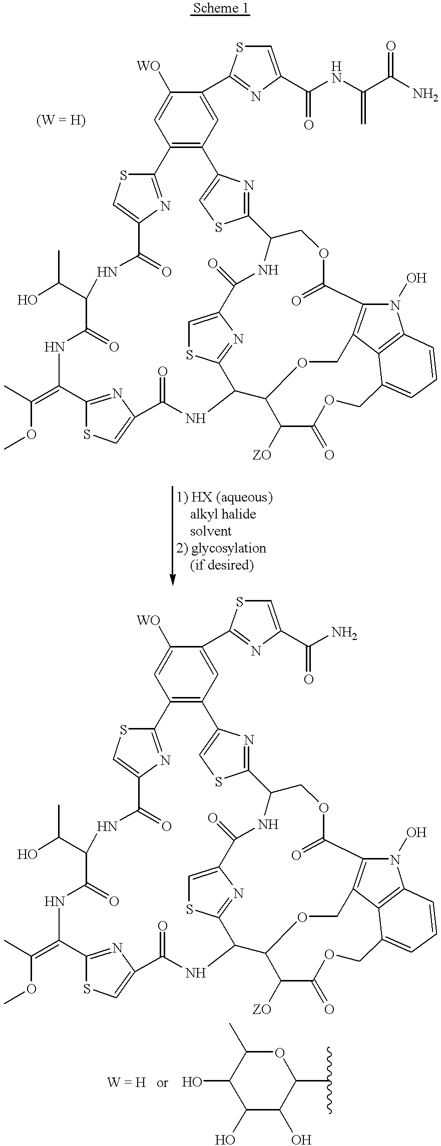

### Analytical Instrumentation

Low resolution MS measurements were performed with a Finnigan SSQ 7000 single quadrupole mass spectrometer, using the positive electrospray ionization mode. MS/MS measurements were conducted in the positive electrospray ionization mode with a Finnigan TSQ 7000 tandem quadrupole mass spectrometer using Argon collision gas or a Finnigan LCQ ion trap mass spectrometer. High resolution MS data were determined with a Finnigan MAT 900 magnetic sector mass spectrometer, positive electrospray ionization mode, ppg reference. The UV spectra were obtained using a Hewlett-Packard 8452A diode array spectrophotometer. IR measurements were taken on a Perkin Elmer 2000 Fourier Transform spectrometer. ¹H-NMR and ¹³C-NMR spectra were obtained on a Bruker DRX-500 instrument operating at 500.13 and 125.76 MHz, respectively, using a Nalorac microprobe. Chemical shifts are reported in ppm relative to solvent (DMSO-d₆, δ_{H}2.49; δ_{C}39.6).

When the nocathiacin compounds herein are employed as pharmaceutical compositions for the treatment of bacterial infections, they may be combined with one or more pharmaceutically acceptable carriers, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 500 mg/kg of animal body weight, preferably given in divided doses two to four times a day, or in sustained release form. For most large mammals the total daily dosage is from about 1 to 100 mg, preferably from about 2 to 80 mg. dosage forms suitable for internal use comprise from about 0.5 to 500 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

These active compounds may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

These active compounds may also be administered parenterally or intraperitoneally. Solutions or suspensions of these active compounds as a free base or pharmacologically acceptable salt can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

The term pharmaceutically acceptable salt includes solvates, hydrates, acid addition salts and quaternary salts. The acid addition salts are formed from a nocathiacin compound having a basic nitrogen and a pharmaceutically acceptable inorganic or organic acid including but not limited to hydrochloric, hydrobromic, sulfuric, phosphoric, methanesulfonic, acetic, citric, malonic, succinic, fumeric, maleic, sulfamic, or tartaric acids. Quaternary salts are formed from a basic nocathiacin compound and an alkyl or arylalkyl halide, preferably methyl or benzyl bromide.

### EXAMPLES

The following examples set out the preparation of novel nocathiacin antibiotic products and their biological properties. Reasonable variations, such as those which would occur to a skilled artisan can be made herein without departing from the scope of the invention.

### Nocathiacin Biotransformation Products

### Example 1

### Synthesis by Biotransformation and Isolation of Nocathiacin IV

To 200 mL of a solution of nocathiacin I in DMF (1 mg/mL) in a 500 mL flask, was added 400 mg of protease from Streptomyces griseus (Sigma, Cat# P5147). The flask was incubated at 27° C. and 200 rpm for 45 hours The reaction mixture from three flasks was pooled and centrifuged (3000 rpm, 15 min). The supernatant was evaporated in vacuo to dryness in a rotary evaporator to yield 0.76 g of brown residue.

10 mL of DMF was added to the brown residue and the insoluble material was removed by centrifugation (13000 rpm, 5 min). The resulting solution was subjected to preparative HPLC using the Beckman System Gold preparative HPLC system with an YMC Pro-C18 column (20 mmID×250 mm length, 5μ particle size, 120 Å pore size). Elution flow rate was 10 mL/min. In each run, sample (1 to 2 mL) was loaded onto the column at 1 mM HCl (solvent A)-acetonitrile (solvent B) 70/30 v/v and separated using the following gradient program: 30%B, 12 min; 30% to 35%B(or 34% or 33% B) linear gradient, 1 min; 35%(or 34% or 33% B), 30 min. Detection (UV) was at 330 nm. The fractions containing nocathiacin IV were assayed with analytical HPLC and pooled. The nocathiacin IV solution was evaporated in vacuo to a small volume, then was frozen and lyophilized. A total of 258 mg of nocathiacin IV was obtained as the hydrochloride salt.

### Physico-Chemical Properties of Nocathiacin IV (Hydrochloride Salt)

Description: shiny yellow granular powder

Molecular Formula: C₅₈H₅₇N₁₃O₁₇S₅-HCl

Molecular Weight: 1367

Formula Weight: 1403

Mass Spectrum: HR-ESIMS [M+H]⁺m/z 1368.26927 ESI-MS/MS fragmentation ions: m/z 1197, 1179, 1135

Infrared Spectrum: Major IR Bands (cm⁻¹) 3427, 1650, 1536, 1474, 1208, 1128, 604.

Ultraviolet Spectrum: λₘₐₓ (MeOH) nm 221, 294, 359 (log ε 4.84,4.45, 4.22).

Circular Dichroism: CD λ nm (Δε) (MeOH) 357(+6.3), 306(−7.4), 266(+23.7), 239(−51.8).

HPLC (Rt) 8.8 min; (as described in the Analytical HPLC section).

¹H-NMR Observed Chemical Shifts (relative to DMSO-d₆ signal δ2.49):

δ10.79 (1H, s), 9.08 (1H, s), 8.62 (1H, s), 8.57 (1H, br), 8.55(1H br), 8.51 (1H, s), 8.44 (1H, s), 8.22 (1H, s), 7.98 (1H, s), 7.86 (1H, s), 7.84 (1H, m), 7.72 (1H, d, J=8.4 Hz), 7.67 (1H, s), 7.34 (2H, br), 7.18 (1H, d, J=6.6 Hz), 6.50 (4H, s), 6.00 (1H, d, J=12.0 Hz), 5.72 (2H, m), 5.21 (1H, m), 5.05 (1H, br), 5.03 (1H, s), 4.96 (1H, d, J=5.3 Hz), 4.78 (1H, d, J=10.2 Hz), 4.52 (1H, d, J=10.9 Hz), 4.29 (1H, d, J=9.6 Hz), 4.24 (1H, m), 4.13 (1H, d, J=10.5 Hz), 4.03 (1H, d, J=9.3 Hz), 3.89 (3H, s), 3.87 (1H, m), 3.07 (1H, br), 2.86 (6H, s), 2.47 (1H, m), 2.11 (1H, br), 1.98 (3H, s), 1.92 (1H, d, J=10.2 Hz), 1.59 (3H, s), 1.51 (1H, m), 1.14 (3H, br), 0.79 (3H, d, J=6.4 Hz).

¹³C-NMR Observed Chemical Shifts (relative to DMSO-d₆ signal δ39.6):

δ171.6, 168.2, 168.0, 167.8, 167.0, 163.7, 163.3, 161.7, 161.6, 161.1, 160.6, 160.4, 158.9, 154.3, 151.1, 150.8, 149.7, 148.8, 145.6, 143.3, 135.0, 134.4, 130.3, 128.0, 127.6, 126.9, 126.4, 126.3, 125.8, 125.7, 124.0, 123.2, 120.0, 119.4, 112.9, 111.2, 109.6, 94.7, 79.2, 71.0, 68.9, 67.7, 66.8, 65.3, 64.6, 63.2, 63.1, 56.2, 55.5, 50.1, 50.0, 46.4, 44.0, 38.9, 30.3, 17.9, 17.6, 13.1.

### Example 2

### Synthesis of Nocathiacin IV by Chemical Methods

A suspension of nocathiacin 1 (3.1 g, 2.1 mmol) in THF (10 mL) was treated with hydroiodic acid (57% in water, 0.5 mL, 3.8 mmol) and methyl iodide (1.0 mL, 16 mmol) The reaction mixture was heated in a sealed tube at 45° C. for 16 h. The reaction mixture was then allowed to cool to room temperature. Diethyl ether (25 mL) was then added to the mixture, and the resulting yellow precipitate was collected by filtration, washed with diethyl ether (3×25 mL) and dried under reduced pressure to afford 3.4 g of crude material containing Nocathiacin IV (82% purity) as the HI salt (88% crude yield).

A portion of the crude nocathiacin IV (517 mg) was purified by reverse phase chromatography on a preparative C-18 column using CH₃CN/H₂O/TFA as mobile phase (gradient elution 20% CH₃CN/78% H₂O/2% TFA to 35% CH₃CN/63% H₂O/2% TFA). Nocathiacin IV as the TFA salt was isolated (180 mg, 93% pure). This material was used for characterization and comparison with the biotransformation product.

### Physico-Chemical Properties of Nocathiacin IV (Trifluoroacetic acid salt)

| Description: | yellow granular powder |
|---|---|
| Molecular | |
| Formula: | C₅₈H₅₇N₁₃O₁₇S₇-TFA |
| Molecular | |
| Weight: | 1367 |
| Formula | |
| Weight: | 1482 |
| Mass Spectrum: | HR-ESIMS [M + H]⁺ m/z 1368.269 |
| | ESI-MS/ MS fragmentation ions: m/z 1197, |
| | 1179, 1153, 1135, 1117, 719 |
| Infrared Spectrum: | Major IR Bands (cm-⁻¹) 3438, 1676, 1536, 1475, |
| | 1204, 1132, 596 |
| Ultraviolet Spectrum: | λₘₐₓ (MeOH) nm 219, 294, 359 |
| Circular Dichroism: | CD λ nm (Δε) (MeOH) 3.55 (+5.6), 305 (−6.3), |
| | 265.5 (+21.0), 239 (−43.5), 210.5 (+29.9) |
| HPLC (Rt): | 8.8 min: (as described in the Analytical HPLC |
| | section). A sample of this semi-synthetic |
| | material was co-injected with a sample of the |
| | biotransformation product and they had |
| | identical retention times. |
| ¹H-NMR: | Observed Chemical Shifts (relative to DMSO- |
| | d₆ signal δ 2.50): |
| | δ 10.84 (1H, s), 10.78 (1H, s), 9.11 (1H, s), 8.65 |
| | (1H, s), 8.59 (1H, br), 8.57 (1H, br), 8.54 (1H, s), |
| | 8.46 (1H, s), 8.22 (1H, s), 7.99 (1H, s), 7.89 (1H, |
| | s), 7.86 (1H, d, J = 11 Hz), 7.75 (1H, d, J = 8.5), |
| | 7.71 (1H, s), 7.37 (2H m), 7.19 (1H, d, J = 7.0 |
| | Hz), |
| | 6.02 (1H, d, J = 12.0 Hz), 5.76 (1H, dd, J = 11.2 |
| | Hz, 4.2 Hz), 5.72 (1H, d, J = 10 Hz), 5.23 (1H, |
| | m), 5.05 (3H, m), 4.79 (1H, d, J = 10.5), 4.53 |
| | (1H, d, J = 11 Hz), 4.30 (1H, d, J = 9.5 Hz), 4.25 |
| | (1H, m), 4.16 (3H, d, J = 0.5 Hz), 4.05 (1H, dd, |
| | J = 9.5 Hz, 1.5 Hz), 3.91 (1H, s), 3.87 (1H, s), |
| | 3.13 (1H, br), 2.88 (6H, m), 2.50 (1H, br), 2.12 |
| | (1H, m), 2.0 (3H, s), 1.94 (1H, d = 14.5 Hz), |
| | 1.60 (3H, s), 1.52 (1H, d, J = 7 Hz), 1.17 |
| | (3H, br), 0.8 (3H, d, J = 7.0 Hz) |
| ¹³C-NMR | Observed Chemical Shifts (relative to DMSO- |
| | d₆ signal δ 39.6) |
| | δ 171.3, 168.0, 167.8, 167.6, 166.8, 163.6, 163.1, |
| | 161.4, 160.9, 160.4, 160.2, 158.7, 154.1, 150.9, |
| | 150.6, 149.5, 148.6, 145.39, 143.1, 134.8, 134.2, |
| | 130.1, 127.8, 127.4, 126.7, 126.3, 126.1, 125.6, |
| | 125.5,123.8, 123.0, 119.8, 119.3, 112.7, 111.0, |
| | 109.4, 94.5, 78.9, 72.2, 70.9, 68.8, 67.5, 66.3, |
| | 65.1, 64.4, 63.0, 62.7, 56.0, 49.9, 49.7, 46.5, |
| | 42.1, 38.1, 30.0, 17.7, 17.3, 12.9 |

### Example 3

### Synthesis of Nocathiacin IV (free base)

To a solution of nocathiacin IV-TFA salt (compound of Example 2, 35 mg) in THF/CH₃CN (3.5 mL, 6:1) was added 1,3-2-diazaphosphorine on polystyrene (26 mg, 2.3 mmol/g) and the mixture was stirred at 25° C. for 30 minutes. The resin-bound base (1,3-2-diazaphosphorine on polystyrene) was then removed by filtration. The filter cake was washed with MeOH, THF, CH₃CN and H₂O. The filtrate was then concentrated under reduced pressure in order to remove volatiles. The resulting solution was frozen and lyophilized to afford 24 mg of nocathiacin IV as the free base.

### Physico-Chemical Properties of Nocathiacin IV (free base)

| | |
|---|---|
| Description: | yellow granular powder |
| Molecular Formula: | C₅₈H₅₇N₁₃O₁₇S₅ |
| Molecular Weight: | 1367 |
| Formula Weight: | 1367 |
| Mass Spectrum: | HR-ESIMS [M + H]⁺ m/z 1368.267 |
| | ESI-MS/MS fragmentation ions: m/z 1368.0, |
| | 1196.9, 1153.1, 1134.8, 1116.9, 719.1 |
| HPLC (Rt): | 8.8 min: (as described in the Analytical HPLC |
| | section). A sample of the free base had the |
| | same retention time as the semi-synthetic |
| | material (compound of Example 2) and with a |
| | sample of the biotransformation product |
| | (compound of Example 1). |
| ¹H-NMR: | Observed Chemical Shifts (relative to DMSO- |
| | d₆ signal δ 2.50): |
| | δ 10.74 (1H, s), 9.10 (1H, s), 8.65 (1H, br), 8.60 |
| | (1H, s) 8.58 (1H, br), 8.51 (1H, s), 8.40 (1H, s), |
| | 8.25 (1H, s), 7.99 (1H, s), 7.89 (1H, s), 7.88 (1H, |
| | d, J = 11.5 Hz), 7.73 (1H, m), 7.70 (1H, br), 7.37 |
| | (2H m), 7.18 (1H, d, J = 7.0 Hz), 6.00 (1H, d, |
| | J = 12.0 Hz), 5.74 (1H, dd, J = 11.0 Hz, 4.5 Hz), |
| | 5.69 (1H, d, J = 8 Hz), 5.22 (1H, m), 5.05 (2H, |
| | m), 5.00 (1H, m), 4.77 (1H, d, J = 10.5), 4.52 (1H, |
| | d, J = 11 Hz), 4.30 (1H, d, J = 9.5 Hz), 4.26 (1H, |
| | s), 4.15 (1H, m), 4.03 (1H, m), 3.98 (3H, br), 3.80 |
| | (1H, m), 3.47 (1H, m), 3.18 (1H, m), 2.52 (6H, |
| | m), 2.23 (1H, m), 1.99 (3H, s), 1.83 (2H, m), 1.63 |
| | (1H, m), 1.45 (3H, s), 1.32 (1H, m), 1.17 (3H, br), |
| | 0.85 (3H, d, J = 7.5 Hz). |

### Example 4

### Synthesis and Isolation of Nocathiacin IV-6-deoxyglucoside

From the frozen vegetative stock culture of Actinoplanes sp. ATCC 53771, 2 mL was used to inoculate 100 mL of seed medium containing the following per liter of deionized water: dextrin, 10 g; glucose, 1 g; beef extract, 3 g; Aradamine pH, 5 g; NZ Amine Type E, 5 g; potassium phosphate, 0.37 g; calcium carbonate, 0.5 g; magnesium sulfate, 0.05 g, in a 500 mL flask. The culture was incubated at 28° C. on a rotary shaker operating at 250 rpm for 2 days. 10 mL of the resulting culture was used to inoculate each of the sixteen 500 mL flasks containing the medium with the following per liter of deionized water: Glucose, 10 g; HY-case SF, 2 g; beef extract, 1 g; corn steep liquor, 3 g. The cultures were incubated at 28° C. on a rotary shaker operating at 250 rpm for 24. hours. The culture was pooled and centrifuged at 3000 rpm for 5 min. The supernatant was discarded and 120 mL of potassium phosphate buffer (100 mM, pH 6.8) containing 50 g/L dextrose was added to the cell to give about 200 mL of cell suspension, which was transferred into two 500 mL flasks (100 mL per flask). 78 mg nocathiacin IV hydrochloride salt was dissolved in 1 mL DMF and was delivered into the two flasks (0.5 mL per flask). The flasks were incubated at 28° C. and 230 rpm for 21 hours. The reaction mixture was pooled and centrifuged at 3000 rpm for 15 min and supernatant was discarded. The cell was extracted with DMF twice (100 mL each time). The DMF extract was pooled and evaporated in vacuo to dryness in a rotary evaporator to about 5.4 mL.

The 5.4 mL concentrated extract was centrifuged (13000 rpm, 5 min) to remove insoluble material. The resulting clear solution was subjected to preparative HPLC using the Beckman System Gold preparative HPLC system with an YMC ODS-AQ column (20 mm ID×150 mm length, 5μ particle size, 120 Å pore size). Elution flow rate was 10 mL/min. Sample (about 1 mL) was loaded onto the column at 1 mM HCl (solvent A)-acetonitrile (solvent B) 85/15 v/v and separated using the following gradient program: 15% B, 3 min; 15% to 30% B linear gradient, 5 min; 30% B, 2 min; 30% to 33% B linear gradient, 1 min; 33% B, 7min; 33% to 40%B linear gradient, 3 min; 40% B, 9 min. Detection (UV) was at 330 nm. Two well resolved peaks were collected and assayed with analytical HPLC. The first peak ( between 14 to 16.5 min) contained pure nocathiacin IV-6-deoxyglucoside. The second peak (between 24 to 27 min) contained nocathiacin IV with small amount of impurity. The nocathiacin IV-6-deoxyglucoside fractions were pooled and evaporated in vacuo to a small volume, then was frozen and lyophilized to yield 26.1 mg of nocathiacin IV-6-deoxyglucoside hydrochloride salt. The nocathiacin IV fractions were pooled and evaporated in vacuo to a small volume then was frozen and lyophilized to recover 17.3 mg of nocathiacin IV hydrochloride salt.

### Physico-Chemical Properties of Nocathiacin IV 6-Deoxyglucoside (Hydrochloride Salt)

| Description: | yellow spongy amorphous solid |
|---|---|
| Molecular Formula: | C₆₄H₆₇N₁₃O₂₁S₅-HCl |
| Molecular Weight: | 1513 |
| Formula Weight: | 1549 |
| Mass Spectrum: | HR-ESIMS [M + H]⁺ m/z 1514.32553 |
| | ESI-MS/MS fragmentation ions: m/z 1368, |
| | 1343, 1197, 917 |
| Infrared Spectrum: | Major IR Bands (cm⁻¹) 3391, 2935, 2360, 2342, |
| | 1664, 1537, 1474, 1420, 1386, 1252, 1210, 1130, |
| | 1089, 1002, 884, 790, 756, 670. |
| Ultraviolet | λₘₐₓ (MeOH) nm 222, 294, 347 (log ε 4.78, 4.39, |
| Spectrum: | 4.20). |
| Circular Dichroism: | CD λ nm (Δε) (MeOH) 346(+5.3), 304(−6.2), |
| | 267(+12.5), 256(+14.0), 239(−16.7), 223(−16.4). |
| HPLC (Rt) | 7.5 min; (as described in the Analytical HPLC |
| | section) |
| ¹H-NMR | Observed Chemical Shifts (relative to DMSO- |
| | d₆ signal δ 2.49): |
| | δ 10.80 (1H, s), 9.08 (1H, s), 8.79 (1H, br), 8.63 |
| | (1H, s), 8.56 (1H, d, J = 8.1), 8.51 (1H, s), 8.47 |
| | (1H, s), 8.23 (1H, s), 8.22 (1H, s), 7.91 (1H, s), |
| | 7.85 (1H, d, 1 = 11.1 Hz), 7.72 (1H, d, J = 8.4 |
| | Hz), 7.69 (1H, s), 7.45 (1H, s), 7.35 (2H, m), 7.18 |
| | (1H, d, J = 7.0 Hz), 6.50 (4H, br), 6.01 (1H, d, |
| | J = 12.1 Hz), 5.98 (1H,s), 5.72 (2H, m), 5.22 |
| | (1H, m), 5.05 (2H, m), 4.96 (1H, d, J = 8.0 Hz), |
| | 4.78 (1H, d, J = 10.7 Hz), 4.54 (1H, d, J = 10.9 |
| | Hz), 4.29 (1H, d, J = 6.6 Hz), 4.23 (1H, m), 4.14 |
| | (1H, d, J = 10.4 Hz), |
| | 4.09 (1H, s), 4.03 (1H, d, J = 8.9 Hz), 3.94 (1H, |
| | dd, J₁ = 9.3 Hz, J₂ = 3.0 Hz), 3.89 (3H, m), 3.88 |
| | (1H, m), 3.61 (1H, m), 3.38 (1H, t, J = 9.3 Hz), |
| | 3.10 (1H, s), 2.88 (6H, s), 2.49 (1H, br), 2.11 (1H |
| | m), 1.99 (3H, s), 1.92 (1H, d, J = 14.3 Hz), 1.60 |
| | (3H, s), 1.21 (1H, m), 1.19 (3H, d, J = 6.1 Hz), |
| | 1.13 (3H, br), 0.80 (3H, d, J = 6.7 Hz). |
| ¹³C-NMR | Observed Chemical Shifts (relative to DMSO- |
| | d₆ signal δ 39.6): |
| | δ 171.4, 168.2, 167.7, 166.9, 164.1, 163.7, 163.1, |
| | 162.2, 161.4, 161.0, 160.5, 160.2, 158.9, 154.0, |
| | 151.5, 149.7, 148.8, 148.7, 145.6, 144.3, 139.3, |
| | 135.0, 129.3, 129.1, 127.9, 126.9, 126.8, 126.5 |
| | 126.4, 125.6, 125.5, 123.9, 123.1, 120.4, 119.4, |
| | 112.7, 111.0, 109.7, 99.6, 94.6, 79.2, 71.8, 71.0, |
| | 70.5, 70.3, 69.8, 69.0, 67.5, 67.2, 66.8, 65.2, |
| | 64.5, 63.2, 63.1, 56.1, 55.5, 49.9, 49.8, 46.0, |
| | 42.9, 38.4, 30.3, 18.0, 17.7, 17.5, 13.0. |

### Biological Evaluation of Nocathiacin Derivatives

### Example 5

### Antibiotic Activity of Nocathiacin IV and Nocathiacin IV 6-deoxyglucoside

To demonstrate its antimicrobial properties, the minimum inhibitory concentration (MIC) for compounds of the invention was obtained against a variety of bacteria using a conventional broth micro dilution assay in accordance with standards recommended by the National Committee for Clinical Laboratory Standards (NCCLS). The serial broth dilution method used Mueller-Hinton medium except for the Streptococcus pneumoniae which was tested in 50% Mueller-Hinton medium and 50% Todd Hewitt medium. The final bacterial inoculate contained approximately 5×10⁵ cfu/well and was run on microtiter plates. The volume of each well was 100 μL and the plates were incubated at 35° C. for 18 hours in ambient air. The MIC was defined as the lowest drug concentration that prevented visible growth. Some of the results obtained are shown in Table 1 below, and demonstrate that compounds of this invention have utility in treating bacterial infections.

TABLE 1

| | | | MIC (ug/ml) |
|---|---|---|---|
| | | MIC (μg/ml) | Nocathiacin IV 6- |
| | | Nocathiacin | deoxyglucoside |
| Organism | Strain | IV (HCI salt) | (HCI salt) |
| Streptococcus pneumoniae | A9585 | 0.001 | 0.03 |
| Streptococcus | A27881 | 0.001 | 0.03 |
| pneumoniae/peniciliin | | | |
| intermediate | | | |
| Streptococcus | A28272 | 0.001 | 0.03 |
| pneumoniae/penicillin | | | |
| resistant | | | |
| Enterococcus faecalis | A20688 | 0.03 | 0.25 |
| Enterococcus faecalis + | A20688 | 0.03 | 0.25 |
| 50% calf serum | | | |
| Enterococcus faecium | A24885 | 0.06 | 0.5 |
| Enterococcus | A28142 | 0.03 | 0.5 |
| faecium/vancomycin | | | |
| resistant | | | |
| Staphylococcus aureus/β | A15090 | 0.03 | 0.5 |
| lactamase positive | | | |
| Staphylococcus aureus + | A15090 | 0.03 | 0.25 |
| 50% calf serum | | | |
| Staphylococcus aureus/ | A27223 | 0.03 | 0.5 |
| homo methidillin resistant | | | |
| Staphylococcus aureus + | A27223 | 0.03 | 0.25 |
| 50% calf serum | | | |
| Staphylococcus epidermidis | A24548 | 0.06 | 0.25 |
| Staphylococcus | A27298 | 0.06 | 0.5 |
| haemolyticus | | | |
| Staphylococcus | A9497 | 0.03 | 0.5 |
| aureus/209P/ATCC6538P | | | |
| Bacillus subtilis/ATCC6633 | A9506A | 0.06 | 0.5 |
| Moraxella catarrhalis/β- | A22344 | 0.25 | 2 |
| lactamase positive | | | |

### Example δ

### Nocathiacin IV and Nocathiacin IV 6-deoxyglucoside in vivo Antibiotic Activity in a Systemic Staphylococcus aureus Infection Model

Nocathiacin IV and nocathiacin IV 6-deoxyglucoside were evaluated for antibiotic activity in vivo, in a systemic infection model using female ICR mice. The animals were infected intraperitoneally (IP) with 6.5×10⁶ CFU of an overnight culture of Staphylococcus aureus A15090 suspended in 7% mucin. The compound was dissolved in a test formulation consisting of 10% DMSO, 5% Tween 80 and 85% water. The solution was administered subcutaneously (SC) at 100 mg/kg total dose (2×50 mg/kg doses at 1 and 4 hours post-infection). The PD₅₀ of nocathiacin IV was determined to be 1.07 mg/kg. The PD₅₀ of nocathiacin IV 6-deoxyglucoside was determined to be 6.4 mg/kg.

We claim:

1. A compound of Formula I 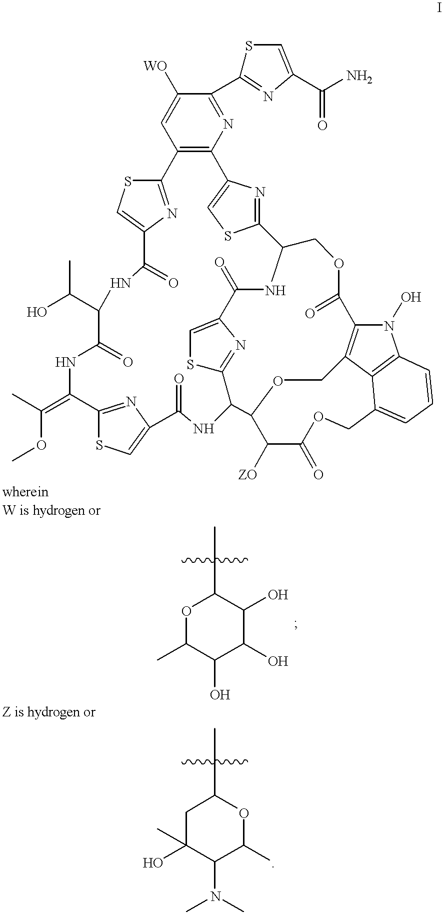
wherein
W is hydrogen or 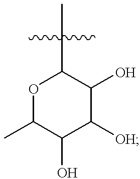
Z is hydrogen or

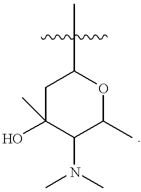

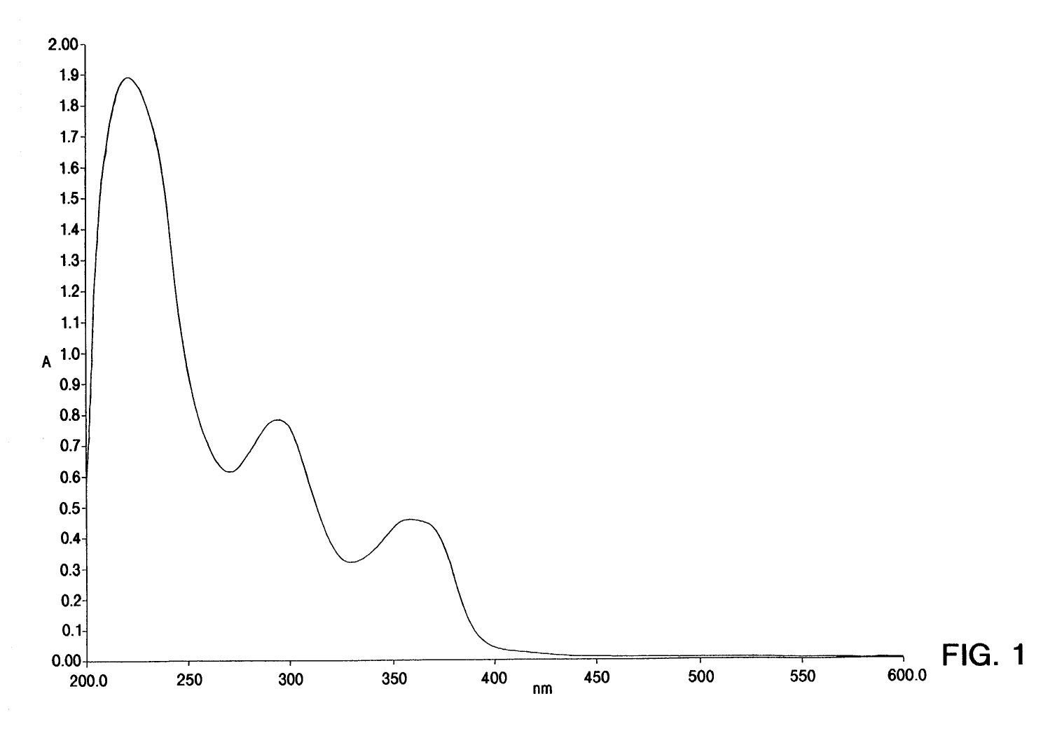

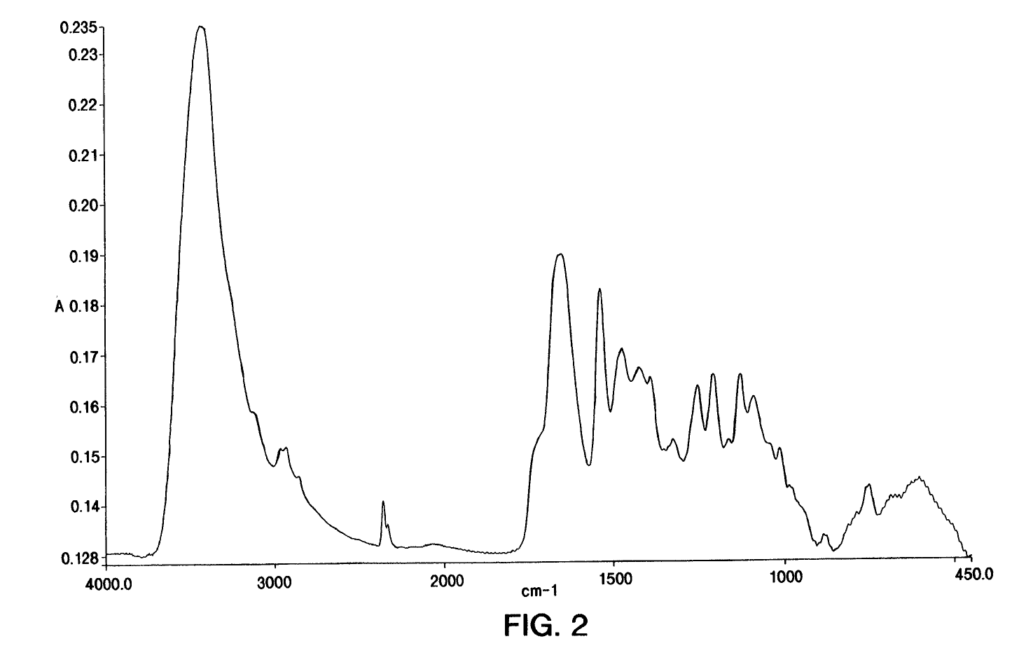

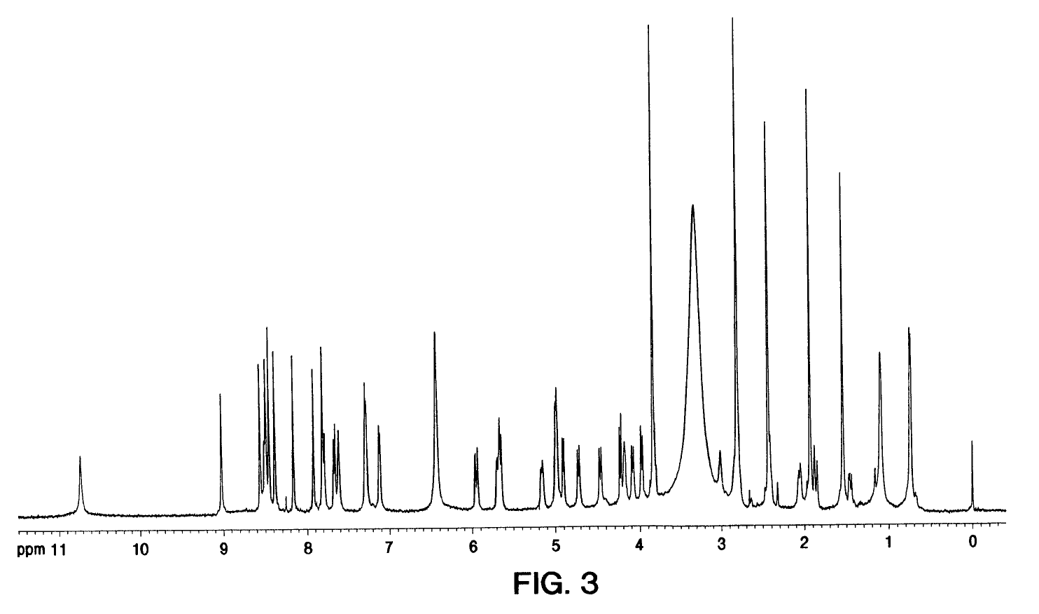

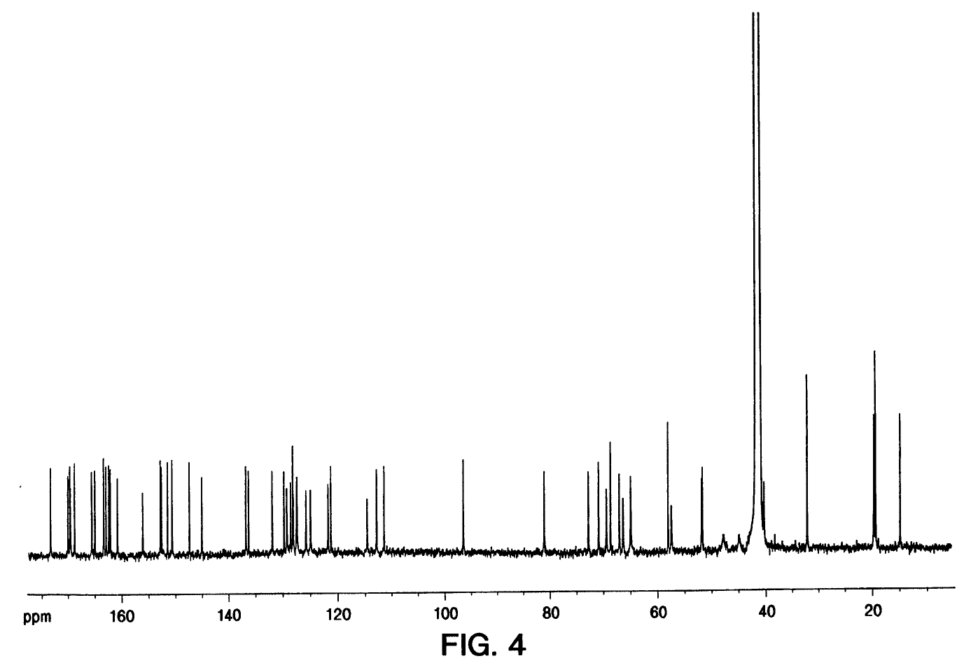

2. An isolated and substantially pure form of nocathiacin IV compound, or a pharmaceutically acceptable salt thereof, wherein the compound has the following characteristics:
(a) its hydrochloride salt appears as a shiny colored granular solid;
(b) has a molecular weight of 1367 as determined by mass spectrometry;
(c) has the molecular formula C₅₈H₅₇N₁₃O₁₇S₅
(d) its hydrochloride salt exhibits an ultraviolet absorption spectrum when dissolved in methanol substantially as shown in FIG. 1;
(e) its hydrochloride salt exhibits an infrared absorption spectrum (KBr) substantially as shown in FIG. 2;
(f) when dissolved in deuterated dimethylsulfoxide, its hydrochloride salt exhibits a proton magnetic resonance spectrum substantially as shown in FIG. 3;
(g) when dissolved in deuterated dimethylsulfoxide, its hydrochloride salt exhibits a ¹³C magnetic resonance spectrum substantially as shown in FIG. 4;
(h) exhibits a high performance liquid chromatography retention time of 8.8 minutes with a C18 reversed phase silica gel column using a 1 mM hydrochloric acid—acetonitrile gradient as described in the analytical HPLC section;
(i) and has the formula

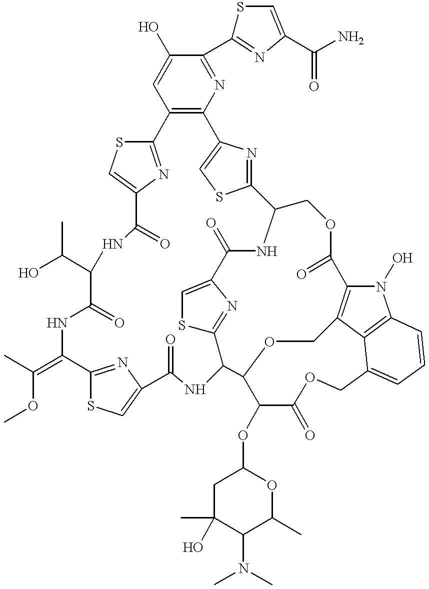

3. The compound of claim 2 wherein the pharmaceutically acceptable salt is the hydrochloride salt.

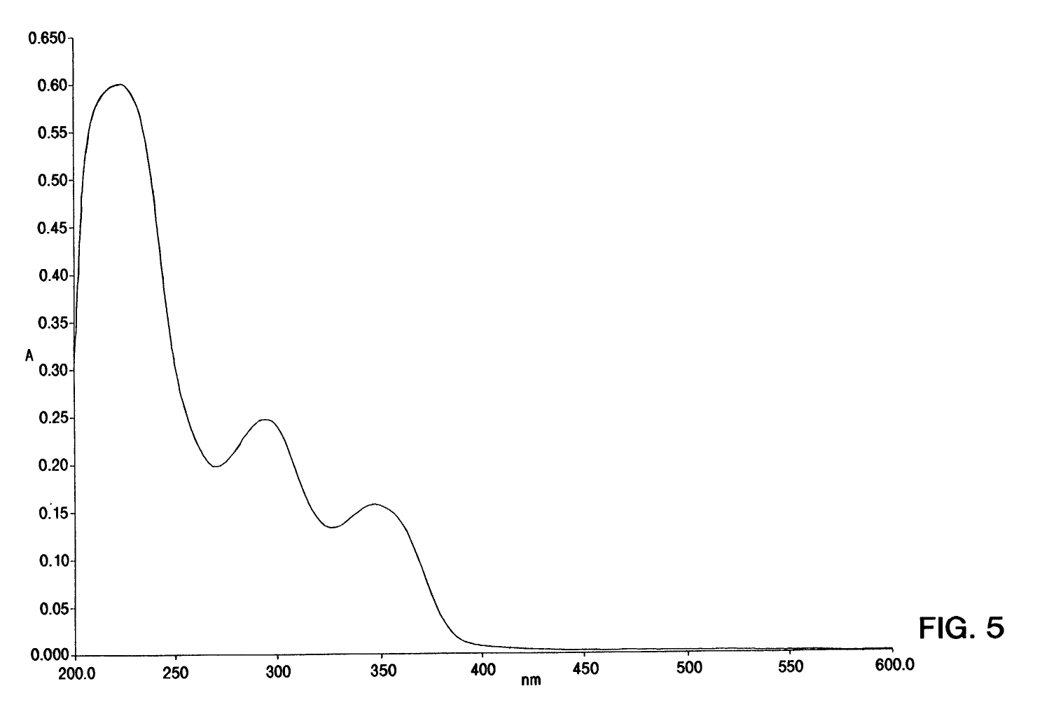

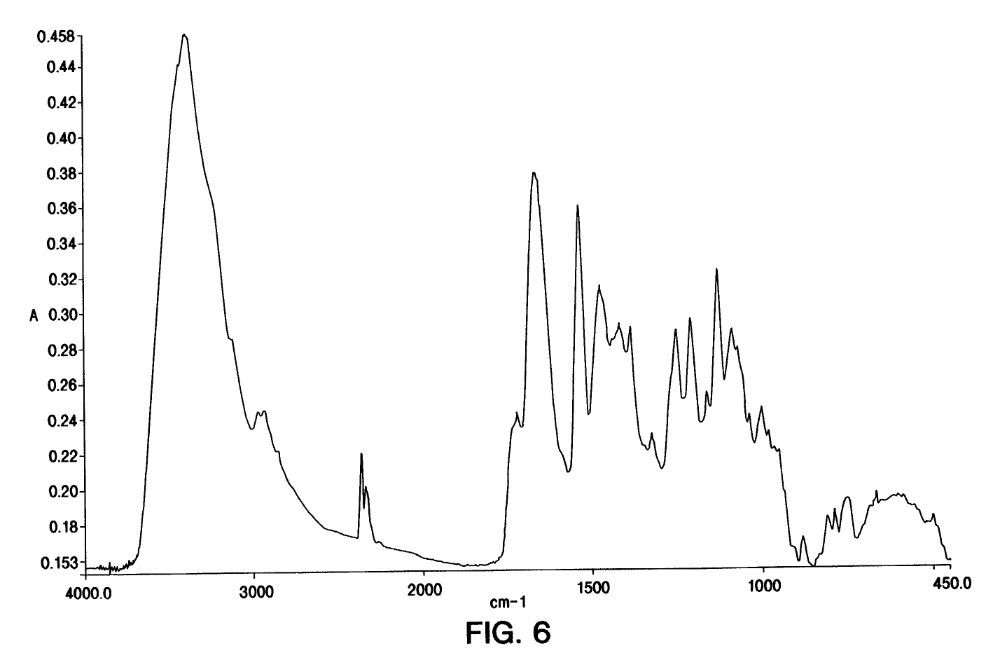

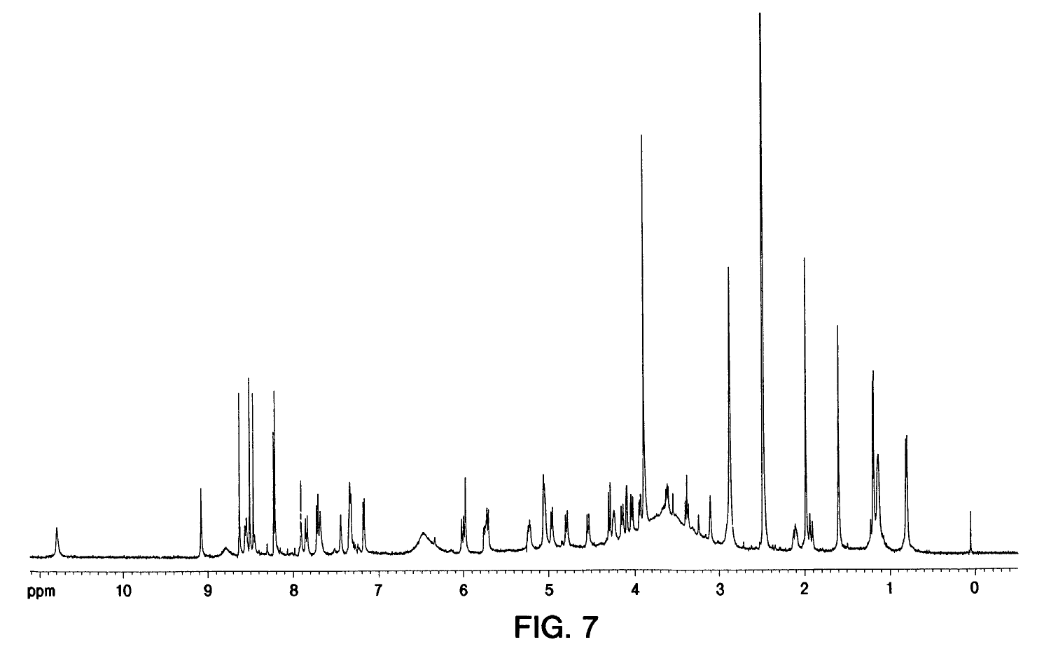

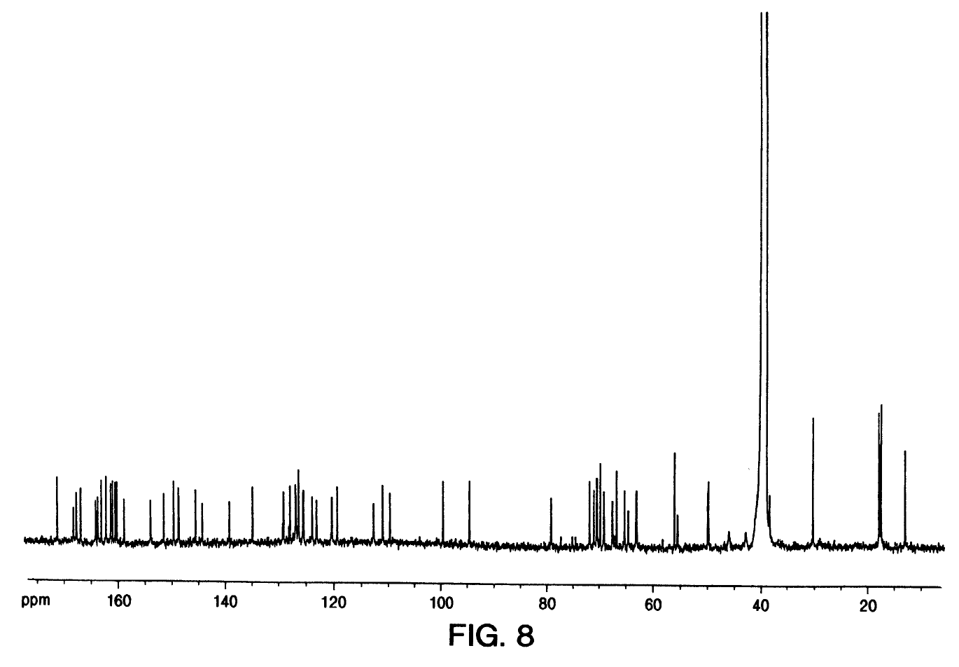

4. An isolated and substantially pure form of nocathiacin IV 6-deoxyglucoside compound, or a pharmaceutically acceptable salt thereof, wherein the compound has the following characteristics:
(a) its hydrochloride salt appears as a spongy colored amorphous solid;
(b) has a molecular weight of 1513 as determined by mass spectrometry;
(c) has the molecular formula C₆₄H₆₇N₁₃O₂₁S₅
(d) its hydrochloride salt exhibits an ultraviolet absorption spectrum when dissolved in methanol substantially as shown in FIG. 5;
(e) its hydrochloride salt exhibits an infrared absorption spectrum (KBr) substantially as shown in FIG. 6;
(f) when dissolved in deuterated dimethylsulfoxide, its hydrochloride salt exhibits a proton magnetic resonance spectrum substantially as shown in FIG. 7;
(g) when dissolved in deuterated dimethylsulfoxide, its hydrochloride salt exhibits a ¹³C magnetic resonance spectrum substantially as shown in FIG. 8;
(h) exhibits a high performance liquid chromatography retention time of 7.5 min with a C18 reversed phase silica gel column using a 1 mM hydrochloric acid—acetonitrile gradient as described in the analytical HPLC section;
(i) and has the formula

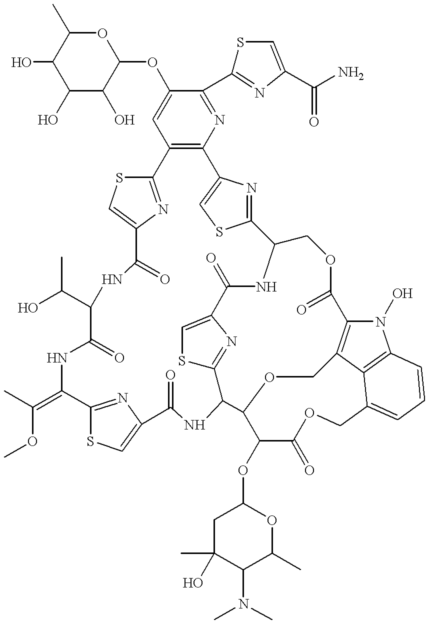

5. The compound of claim 4 wherein the pharmaceutically acceptable salt is the hydrochloride salt.

6. A pharmaceutical composition comprising a therapeutically effective amount of a compound as claimed in any of claims 1 through 5 and a suitable carrier, adjuvant or diluent.

7. A method for preventing or treating infection of a mammal by a bacterium, comprising the step of administering a therapeutically effective amount of a compound as claimed in any of claims 1 through 5 to said mammal in need thereof.

8. A method for making nocathiacin IV from nocathiacin I comprising the steps of converting nocathiacin I to nocathiacin IV in a DMF solution containing protease from Streptomyces griseus (Sigma, Cat# P5147), and isolating said nocathiacin IV from the biotransformation reaction mixture.

9. A method for making nocathiacin IV from nocathiacin I comprising the steps of converting nocathiacin I to nocathiacin IV in THF containing hydroiodic acid and methyl iodide, and isolating said nocathiacin IV from the chemical reaction mixture.

10. A method for making nocathiacin IV 6-deoxyglucoside from nocathiacin IV comprising the steps of converting nocathiacin IV to nocathiacin 6-deoxyglucoside in a biotransformation broth containing fermented culture of microorganism Actinoplanes sp. (ATCC-53771); and isolating said nocathiacin IV 6-deoxyglucoside from the biotransformation reaction broth.
